(19) United States
(12) Patent Application Publication
(10) Pub. No.: US 2009/0036441 A1
(43) Pub. Date: Feb. 5, 2009

(54) Indole Derivatives With Antitumor Activity

(75) Inventors: Mario Grugni, Bresso (IT); Mara Cassin, Bresso (IT); Gennaro Colella, Bresso (IT); Sergio De Munari, Bresso (IT); Gianluca Pardi, Bresso (IT); Paolo Pavesi, Bresso (IT)

Correspondence Address: SEED INTELLECTUAL PROPERTY LAW GROUP PLLC, 701 FIFTH AVE, SUITE 5400, SEATTLE, WA, 98104, US

(73) Assignee: Cell Therapeutics Europe S.R.L., Bresso (IT)

(21) Appl. No.: 11/793,875

(22) PCT Filed: Dec. 22, 2005
(86) PCT No.: PCT/EP2005/013886

§ 371 Date: Sep. 16, 2008

(30) Foreign Application Priority Data
Dec. 23, 2004 (IT) MI2004A002476

(51) Int. Cl. C07D 209/90; A61K 31/403; A61K 31/5377; C07D 413/06; A61K 31/496; C07D 401/06; A61K 31/519; A61P 35/00; C07D 487/02

(52) U.S. Cl. 514/232.8; 548/427; 514/411; 544/142; 544/372; 514/253.03; 514/257; 544/247; 544/245

(57) ABSTRACT

3H-Benzo[e]indol-4,5-dione derivatives with antitumor activity, the processes for the preparation thereof and pharmaceutical compositions containing them.

The present invention relates to compounds with antitumor activity and pharmaceutical compositions thereof. More precisely, the invention relates to 3H-benzo[e]indol-4,5-dione derivatives capable of counteracting tumor growth and angiogenesis through inhibition of the interaction between transcription factor HIF-1α and its coactivator p300 thereby preventing the production of Vascular Endothelial cell Growth Factor (VEGF).

### BACKGROUND OF THE INVENTION

Vascular Endothelial Cell Growth Factor plays a key role in the processes of physiological and physiopathological angiogenesis. A number of mechanisms are involved in the regulation of the VEGF gene, among which a fundamental role is played by the tissue oxygen tension, as proved by the reversible increase in VEGF mRNA levels under in vivo and in vitro hypoxia conditions. The increase in the expression of VEGF mRNA is mainly mediated by the transcription factor HIF-1 (hypoxia-inducible factor-1), which binds to a recognition site in the promoter region of the VEGF gene.

A great number of experimental data show that HIF-1 is a global regulator of oxygen homeostasis and that an impaired activity of HIF-1 promotes survival, proliferation, invasion and metastatization of tumoral cells (1). It has been therefore suggested that therapeutical strategies focusing on the inhibition of HIF-1 activity could increase the survival of cancer patients (2).

HIF-1 is a heterodimer consisting of HIF-1α and HIF-1β sub-units, which dimerize and bind to DNA through the bHLH-PAS domain (3). The expression of the HIF-1α subunit is strictly regulated by the tissue oxygen concentration (4) through processes of ubiquitination and proteasome degradation, mediated by the binding of VHL protein to HIF-1α. Such interaction only takes place when HIF-1α has been hydroxylated at the 402 and 564 proline residues. Oxygen is the limiting substrate for prolyl-hydroxylase which modifies HIF-1α (5). The expression of HIF-1α exponentially increases as O₂ concentration decreases and determines the HIF-1 global activity levels.

The function of HIF-1α transactivation domain is also subject to negative regulation, controlled by oxygen partial pressure. The N-terminal transactivation domain is negatively regulated through the recruitment of hystone deacylase by VHL and by the factor inhibiting HIF-1 (FIH-1), which binds to both VHL and HIF-1α (6).

HIF-1 activation takes place through the presence of p300/CBP coactivators which physically interact with the activation of the HIF1 domain to promote the transcription of genes like VEGF (7). Both p300 and CBP are co-activators also for other transcription factors, such as Stat-3, NF-κB, p53.

The interaction of p300/CBP with HIF-1 is essential to transcription, and recent publications have proved the importance of the HIF-1/p300 interaction for tumor growth (8). HIF-1α C-terminal trans-activation domain (C-TAD) binds to a p300 and CBP domain known as CHI. The binding of CBP and p300 to HIF-1α is negatively regulated through oxygen-dependent hydroxylation of asparagine 803 in the C-terminal activation domain by FIH-1. Thus, hypoxia causes both stabilization to proteasome degradation and transcriptional activity of HIF-1.

Structural details of the interaction between HIF-1α TAD-C and the CH1 domain of p300 or CBP have been elucidated (9, 10). Details of the interaction between p300/CBP and the CITED2 protein (also known as p35^{srj}), which is considered a negative regulator of Hif-1α activity (11), have also been published.

HIF-1 activation induces the transcription of a number of genes involved in the production of angiogenic factors, glucose carriers, glycolytic enzymes, survival, migration and invasion factors, which are particularly important for tumor progression.

Aberrant expression of Hif-1α protein was observed in more than 70% human tumors and their metastases and was connected with an increase in vascularization and tumor progression (12-14). In clinical practice, aberrant expression of Hif-1α was associated to therapy failure and mortality increase in a number of tumoral pathologies, such as non-small cells lung carcinoma (15), oropharyngeal squamous cell cancer (16), early-stage cervical cancer (17), head-and-neck cancer (18), mutated p53 ovary cancer (19), oligodendrioglioma (20) and BCL-2 positive esophageal cancer (21).

Various approaches for inhibiting HIF-1 activity have been described in literature. Some of them suggested the use of antisense oligonucleotides for Hif-1α or negative dominant forms of Hif-1α.

Among the pharmacological approaches, Hif-1α activity inhibitors acting through indirect mechanisms have been described, such as PI3K-mTOR inhibitors (22-23) and MEKK (24) inhibitors which act on the transduction of signals controlling Hif-1α activity; inhibitors of HSP90 chaperone protein (25); thioredoxin reductase inhibitors, which act modifying the cell redox state (26); molecules which destabilize microtubules, such as 2-methoxyestradiol (27) and epothilones (28).

Recently, both constitutive and hypoxia-induced inhibition of Hif-1α levels by PX-478 in human tumors transplanted in nude mice (Melphalan N-oxide) was reported. The compound shows marked antitumoral effects. However, the mechanism of action of this compound has yet to be completely clarified (29).

Finally, chaetomin, a dithiodioxopiperazine metabolite of Chaetomium sp fungi, has recently been reported to interfere with the binding of Hif-1α to p300. The compound acts altering the CH1 domain structure of p300, thus preventing its interaction with Hif-1α. Chaetomin administration to tumor-bearing mice inhibits hypoxia-induced transcription in the tumor and tumor growth (30).

### STATE OF THE ART

Khimiya Geterotsiklicheskikh Soedinenii (1989), 611-14 and (1983), (10), 1364-6 describe 3H-benz[e]indole o-quinones and their chemical modification. No biological activity is reported for the described compounds.

3H-benz[e]indole o-quinones obtained by reaction of naphthoquinones with cyclic β-dicarbonyl compounds are described in Zhurnal Organikeskoi Khimii (1985), 21(6), 1315-20. No biological activity is reported for the described compounds.

The antiviral compound 1-phenyl-2-ethoxycarbonyl-4,5-dioxo-4,5-dihydro-3H-benzo[e]indole is described in Chem. & Pharm. Bull (1983), 31(12), 4391-4400.

A 3H-benz[e]indole o-quinone in which a benzoquinone ring is annulated onto the 1,2 positions of benzoindole nucleus is reported in Heteocycles (1982), 19(11), 2019-2025.

3H-benz[e]indole derivatives are prepared in Chemical & Pharm. Bull. (1983), 31(12), 4401-8.

Archives of Biochemistry and Biophysics 429 (2004) 30-41 discloses the compounds 1-acetyl-8-bromo-2-methyl-4,5-dioxo-4,5-dihydro-3H-benzo[e]indole-1-carboxylate, ethyl 8-bromo-2-(bromomethyl-3-methyl-4,5-dioxo-4,5-dihydro-3H-benzo[e]indole-1-carboxylate and ethyl 8-bromo-3-methyl-2-(1-piperidinyl)methyl-4,5-dioxo-4,5-dihydro-3H-benzo[e]indole-1-carboxylate. The compounds are reported to inhibit protein tyrosine phosphatase α (PTPα) in vitro and cell spreading of fibroblasts on a fibronectin substrate. The compounds are taught to be able to generate hydrogen peroxide in cells in response to a reducing agent or reducing enzyme in a manner unregulated and distributed throughout the cell. The authors conclude that these features make the compounds potentially cytotoxic and unlikely clinical candidates.

### DISCLOSURE OF THE INVENTION

It has now been found that certain derivatives of 3H-benzo[e]indol-4,5-dione are capable of inhibiting the interaction between Hif-1α and p300 and prevent VEGF production in tumour cells under hypoxia conditions. In a first aspect, the invention is directed to a method for preventing, inhibiting or blocking angiogenesis in an animal, preferably in humans, by administering to a subject in need thereof a compound of formula (I):

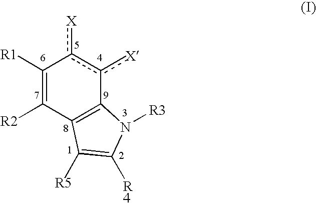

wherein

[symbol] is a single or double bond;

X and X′ are independently 0; OH; NH; NH2; NH2OH;

or X and X′ are nitrogen and, together with the carbon atoms they are linked to, form a 6- or 10-membered heterocyclic or heteroaromatic ring;

R1 and R2, together with the atoms they are linked to (6- and 7-positions in formula (I)), form a 6-membered aromatic or a 5- or 6-membered heteroaromatic ring, preferably a benzene ring optionally substituted with (C1-C4)acyl, (C1-C4)alkylsulfonylamino or (halogen) C1-C4alkyl, halogen, amine, mono or di(C1-C4)alkylamine, hydroxyl, (C1-C4)alkoxyl, thiol, (C1-C4)alkylthiol, carbamoyl, nitrile, sulfamoyl, phenyl;

R3 is hydrogen; acyl(C1-C4), (C1-C4)alkylsulfonyl,

(C1-C4)alkylaminosulfonyl, straight or branched (C1-C4)alkyl, optionally interrupted by —O—, —S—, —N═, —NH—, —NHCONH—, —NHCOO—, —NHSO2NH—, —NHC(═NH)NH—, —NHC(═NH)—, —NHCSNH—, —CO—, —COO—, —CONH—, —SO2-, —SO2NH—, —CH═CH—, —C≡C— groups, or substituted with halogen, —NH2, —OH, —SH, —OCONH2, —COOH, —SO2NH2, —CONH2, —NHCONH2, —CN, phenyl, 5- or 6-membered heterocycle;

R4 is —NR6R7, wherein R6 and R7 are, independently, hydrogen, (C1-C4)acyl, (C1-C4)alkylsulfonyl, (C1-C4)alkylaminosulfonyl, straight or branched (C1-C4)alkyl, optionally substituted with halogen, amine, hydroxyl, thiol, carbamoyl, nitrile, phenyl or a 5- or 6-membered heterocyclic ring, in particular morpholine; —OR6; carbamoyl; straight or branched (C1-C4)alkyl, optionally interrupted by —O—, —S—, —N═, —NH—, —CO—, —COO—, —CONH—, —SO2-, —SO2NH— groups, or substituted with halogen, amine, hydroxyl, thiol, carbamoyl, nitrile, phenyl or a 5- or 6-membered heterocycle; up to 10-membered aromatic or heteroaromatic ring; 5- or 10-membered heterocyclic ring;

R5 is NH2; NR6R7; OR6; straight or branched (C1-C4)alkyl, optionally interrupted by —O—, —S—, —N═, —NH—, —CO—, —COO— groups,

—CONH—, —SO2-, —SO2NH—, or substituted with halogen, amine, hydroxyl, thiol, carbamoyl, nitrile, phenyl or a 5- or 6-membered heterocyclic ring; up to 10-membered aromatic or heteroaromatic ring; a 5 or 6-membered heterocyclic ring; ureido; the salts, isomers, enantiomers or diastereomers thereof.

Particularly preferred are compounds (I) in which X═X′═O (carbonyl groups). Most preferred are the compounds (I) wherein:

R3 is selected from H, methyl, benzyl, carboxymethyl, tert-butoxycarbonylmethyl, carbamoylmethyl;

R4 is a methyl or ethyl group optionally substituted with an hydroxy or amino group or with a primary or secondary amine;

R5 is ethoxycarbonyl.

In biochemical and cellular assays, the compounds of the invention proved capable of inhibiting the interaction between HIF-1α and p300, and the activation of VEGF promoter and the production of secreted VEGF, respectively.

Schemes (1) and (2) illustrate the synthesis of compounds (I) in which X═X′═O and in which, respectively, X and X′ form a diazine.

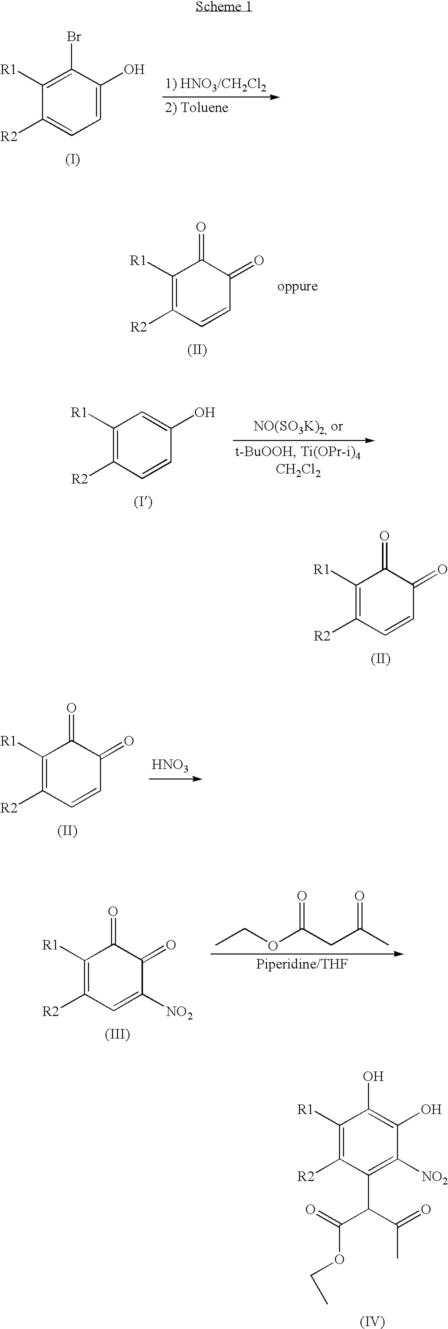

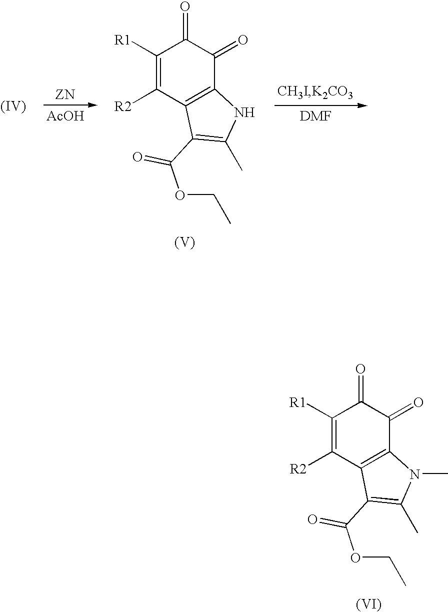

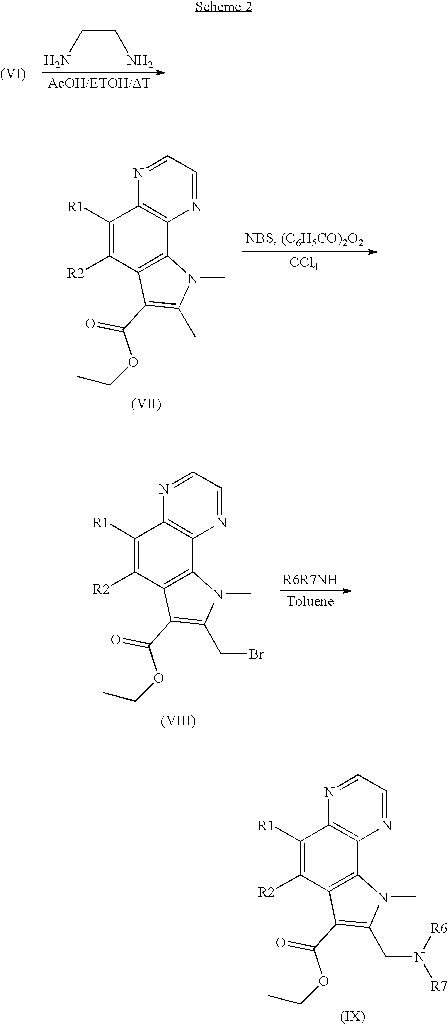

In a another embodiment, the invention provides a 3H-benzo[e]indol-4,5-dione derivative having antitumor activity, which is selected from the group consisting of:
ethyl 8-bromo-3-tert-butoxycarbonylmethyl-2-methyl-4,5-dioxo-4,5-dihydro-3H-benzo[e]indole-1-carboxylate;
ethyl 8-bromo-3-carboxymethyl-2-methyl-4,5-dioxo-4,5-dihydro-3H-benzo[e]indole-1-carboxylate;
ethyl 8-bromo-3-carbamoylmethyl-2-methyl-4,5-dioxo-4,5-dihydro-3H-benzo[e]indole-1-carboxylate;
ethyl 5-bromo-2-methyl-1H-1,8,11-triazacyclopenta[1]phenanthrene-3-carboxylate;
ethyl 5-bromo-2-methyl-1H-1,8,13-triazabenzo[a]cyclopenta[c]anthracene-3-carboxylate;
ethyl 8-bromo-3-methyl-2-(4′-methylpiperazin-1′-yl)methyl-4,5-dioxo-4,5-dihydro-3H-benzo[e]indole-1-carboxylate;
ethyl 8-bromo-3-methyl-2-(piperazin-1′-yl)-methyl-4,5-dioxo-4,5-dihydro-3H-benzo[e]indole-1-carboxylate;
ethyl 8-bromo-3-methyl-2-(4′-(2-hydroxyethyl)-piperazin-1′-yl)-methyl-4,5-dioxo-4,5-dihydro-3H-benzo[e]indole-1-carboxylate;
ethyl 8-bromo-3-methyl-2-(4′-(2-aminoethyl)-piperazin-1′-yl)-methyl-4,5-dioxo-4,5-dihydro-3H-benzo[e]indole-1-carboxylate.

According to a further aspect, the invention relates to pharmaceutical compositions containing an effective amount of at least one of the compounds of formula (I), together with pharmaceutically acceptable excipients. The compositions can be solid, semi-solid or liquid, preferably in the form of solutions, suspensions, powders, granules, tablets, capsules, syrups, suppositories, aerosol or controlled-release systems. The compositions can be administered through different routes, particularly the oral, transdermal, subcutaneous, intravenous, intramuscular, rectal and intranasal routes. The parenteral administration is preferred. Dosages of the active ingredient will be determined by those skilled in the art according to the medical-toxicological and pharmacokinetics characteristics of the specific selected compound, as well as the type, severity and stage of disease to treat, and the weight, sex and age of the patient. A dosage ranging from 0.1 to 100 mg/Kg/day will be generally acceptable.

The amount of active ingredient for unit dosage will depend on the form and route of administration, the compound used, the disease to treat, but as a rule it will generally vary from 0.1 to 1000 mg, preferably 1 to 600 mg.

The principles and methods for the preparation of pharmaceutical compositions are known to those skilled in the art and are described, for example, in Remington's Pharmaceutical Science, Mack Publishing Company, Easton (PA).

In a yet further embodiment, the invention provides a method of treating tumors and metastasis which comprises administering to a subject, preferably a human subject in need of said treatment, an effective amount of a compound of formula (I) or a pharmaceutical composition thereof. Tumors that are preferably treated in accordance with the present invention include lung carcinoma, breast carcinoma, prostate carcinoma, neuroblastoma, glioblastoma multiforme, melanoma, central nervous system tumors, oropharyngeal squamous cell cancer, cervical cancer, ovary, esophageal, kidney, colon, head-and-neck cancers and oligodendrioma.

The invention is further illustrated by the following examples.

### EXAMPLES

### Preparation 1: 6-Bromo-1,2-naphthoquinone

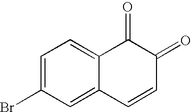

Method A

A solution of 1,6-dibromo-2-naphthol (20 g, 0.0662 moles) in CH₂Cl₂ (200 ml) was added, drop by drop (40 minutes) and under stirring, with 90% HNO₃ (9.39 ml, 0.1988 moles). After completion of the addition, the solution was left under stirring for 15 minutes, then added with H₂O (200 ml). The organic phase was separated, dried over Na₂SO₄ and evaporated to dryness under reduced pressure. The solid residue was suspended in toluene (40 ml) and the mixture was left under stirring at 90° C. for 1 hour. After cooling, the solid was collected, washed with petroleum ether at 40-60° C. and dried under vacuum at 40° C. to give 7.99 g (51% yield) of product (red/orange solid).

¹H NMR (DMSO-d₆): δ 7.90 (1H, d, J=1.79 Hz); 7.84 (1H, d, J=8.21); 7.77 (1H, dd, J=1.79, 8.21); 7.62 (1H, d, J=10.19); 6.46 (1H, d, J=10.19).

Method B

A solution of Fremy's salt (10 g, 0.0373 moles) and KH₂PO₄ (77 g, 0.5658 moles) in H₂O (1.14 l) under nitrogen atmosphere, was added drop by drop and under stirring, with a solution of 6-bromo-2-naphthol (3.032 g, 0.0132 moles) in CH₂Cl₂ (150 ml). After stirring the diphasic system for 21 hours under nitrogen atmosphere, the organic phase was separated, washed with H₂O (3×40 ml), dried over Na₂SO₄ and evaporated to dryness under reduced pressure. The solid residue was suspended in Et₂O (20 ml) and the mixture was left under stirring for 1 hour. The solid was collected and washed with Et₂O and hexane to give 1.253 g (40% yield) of product (brown solid).

Method C

A solution of t-BuOOH in decane (1.1 ml, 0.006 moles), anhydrous CH₂Cl₂ (60 ml) and 4 Å molecular sieves (1 g) was placed in a round-bottom flask under nitrogen atmosphere. In a second round-bottom flask, under nitrogen atmosphere, a solution of 6-bromo-2-naphthol (0.23 g, 0.001 moles) and Ti(OPr-i)₄ (0.31 ml, 0.001 moles) in anhydrous CH₂Cl₂ (50 ml) was prepared. The naphthol—titanium complex was then added, drop by drop (5 hours) and under stirring, to the peroxide solution under nitrogen atmosphere. After completion of the addition, the mixture was left under stirring for one hour, then filtered through a silica gel column. The solvent was evaporated off under reduced pressure and the solid residue collected and dried under vacuum at 50° C. to give 0.043 g (18% yield) of product (brown solid).

### Preparation 2: 6-Bromo-3-nitro-1,2-naphthoquinone

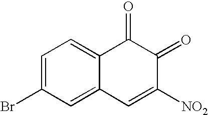

A suspension of 6-bromo-1,2-naphthoquinone (5.2 g, 0.0219 moles) in HNO₃ 70% (10 ml) was kept under stirring at 50° C. for 5 minutes. After addition of ice, the mixture was left under stirring at room temperature for 1 hour. The solid was collected, repeatedly washed with H₂O and dried under vacuum at 40° C. to give 5.81 g (94% yield) of product (red/orange solid).

¹H NMR (DMSO-d6): δ 8.61 (1H, s); 8.18 (1H, d, J=1.37 Hz); 7.95 (2H, m).

### Preparation 3: Ethyl 2-(7-bromo-3,4-dihydroxy-2-nitronaphthalen-1-yl)-3-hydroxybut-2-enoate

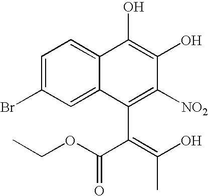

A solution of 6-bromo-3-nitro-1,2-naphthoquinone (9.62 g, 0.0341 moles) in anhydrous THF (60 ml) was added, under stirring, with ethyl acetoacetate (4.78 ml, 0.0375 moles) and piperidine (0.33 ml, 0.003 moles). After completion of the addition, the resulting solution was kept under stirring at room temperature for 1 hour 15 minutes. The solvent was removed under pressure and the oily residue was taken up with Et₂O (150 ml). After removing insolubles, the filtrate was washed with H₂O (2×150 ml) and 10% NaCl (50 ml), dried over Na₂SO₄ and evaporated to dryness under reduced pressure to give 12.98 g (92% yield) of product (dark red oil).

LC-MS: 411.9, [M-H]⁻

¹H NMR (DMSO-d₆) (enol/keto 85:15 mixture; signals of the enol form are reported): δ 13.17 (1H, s); 10.24-9.96 (2H, s); 8.12 (1H, d, J=9.04 Hz); 7.80 (1H, d, J=1.67); 7.69 (1H, dd, J=1.67, 9.04); 4.09 (2H, q, J=7.04); 1.66 (3H, s); 1.01 (3H, t, J=7.04).

### Example 1

### Ethyl 8-bromo-2-methyl-4,5-dioxo-4,5-dihydro-3H-benzo[e]indole-1-carboxylate

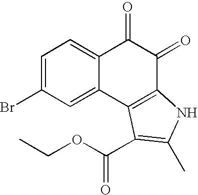

A solution of ethyl 2-(7-bromo-3,4-dihydroxy-2-nitronaphthalen-1-yl)-3-hydroxybut-2-enoate (562 mg, 1.363 mmoles) in glacial AcOH (6 ml) was added, under stirring, with Zn (357 mg, 5.46 mmoles). The resulting suspension was heated at 90° C., under stirring, for 6 hours. After cooling, the solid was collected, suspended in H₂O (12 ml) and the mixture was left under stirring at room temperature for 24 hours. The solid was collected, washed with H₂O, dried under vacuum at 40° C. and suspended in AcOEt (4 ml). The mixture was refluxed for 5 minutes, then cooled, thereafter the solid was collected, washed with AcOEt and dried under vacuum at 40° C. to give 319 mg (yield 65% yield) of product (red solid).

¹H NMR (DMSO-d₆): δ 13.05 (1H, s); 8.79 (1H, d, J=1.83 Hz); 7.78 (1H, d, J=8.26); 7.59 (1H, dd, J=1.83, 8.26); 4.34 (2H, q, J=7.11); 2.46 (3H, s); 1.36 (3H, t, J=7.11).

### Example 2

### Ethyl 8-bromo-2-bromomethyl-3-methyl-4,5-dioxo-4,5-dihydro-3H-benzo[e]indole-1-carboxylate

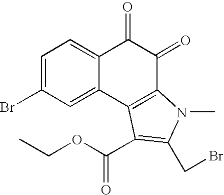

A suspension of ethyl 8-bromo-2,3-dimethyl-4,5-dioxo-4,5-dihydro-3H-benzo[e]indole-1-carboxylate (100 mg, 0.266 mmoles) in CCl₄ (8 ml), kept under stirring at room temperature, was added with N-bromosuccinimide (50 mg, 0.2809 mmoles) and dibenzoyl peroxide (1 mg, 0.004 mmoles). The resulting suspension was heated to reflux for 6 hours under stirring. After cooling, the solid was filtered off and the filtrate evaporated to dryness under reduced pressure. The residue was taken up with Et₂O and the solid was collected to give 59 mg (49% yield) of product (red solid).

¹H NMR (DMSO-d₆): δ 8.40 (1H, d, J=1.79 Hz); 7.82 (1H, d, J=8.29); 7.63 (1H, dd, J=1.79, 8.29); 4.98 (2H, s); 4.42 (2H, q, J=7.08); 3.99 (3H, s); 1.40 (3H, t, J=7.08).

### Example 3

### Ethyl 8-bromo-2,3-dimethyl-4,5-dioxo-4,5-dihydro-3H-benzo[e]indole-1-carboxylate

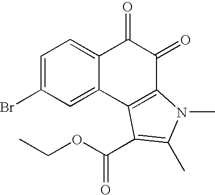

A suspension of ethyl 8-bromo-2-methyl-4,5-dioxo-4,5-dihydro-3H-benzo[e]indole-1-carboxylate of Example 1 (1.43 g, 3.948 mmoles), K₂CO₃ (2.73 g, 19.7525 mmoles) and CH₃I (1.23 ml, 19.7525 mmoles) in dry DMF (140 ml) was heated at 60° C., under stirring and nitrogen atmosphere, for 3 hours. After cooling, the inorganic solid was filtered off and the filtrate was diluted with H₂O (140 ml) and left under stirring at room temperature for 2 hours. The precipitated solid was collected, repeatedly washed with H₂O, dried under vacuum at 40° C. and chromatographed on silica gel using petroleum ether (bp 40-60° C.)/AcOEt 1/1 as the eluent. The resulting solid was suspended in AcOEt (6 ml) and the mixture was heated to reflux for 5 minutes. After cooling, the solid was collected, washed with AcOEt and petroleum ether 40-60° C. and dried under vacuum at 40° C. to give 445 mg (30% yield) of product (red solid).

| | | | | | |
|---|---|---|---|---|---|
| Elemental | Calculated | C 54.27% | H 3.75% | N 3.72% | Br 21.24% |
| analysis | Found | C 54.11% | H 3.72% | N 3.78% | Br 21.08% |

LC-MS: 378.1, MH⁺

¹H NMR (DMSO-d₆): δ 8.31 (1H, d, J=1.84 Hz); 7.78 (1H, d, J=8.23); 7.59 (1H, dd, J=1.84, 8.23); 4.37 (2H, q, J=7.12); 3.90 (3H, s); 2.43 (3H, s); 1.37 (3H, t, J=7.12).

### Example 4

### Ethyl 8-bromo-3-methyl-2-(morpholin-4′-yl)methyl-4,5-dioxo-4,5-dihydro-3H-benzo[e]indole-1-carboxylate

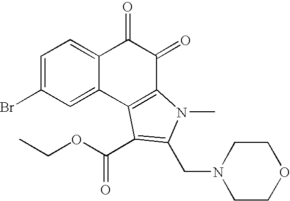

A solution of ethyl 8-bromo-2-bromomethyl-3-methyl-4,5-dioxo-4,5-dihydro-3H-benzo[e]indole-1-carboxylate of Example 2 (86 mg, 0.183 mmoles) and morpholine (32 μl, 0.367 mmoles) in anhydrous toluene (4 ml) was heated at 50° C., under stirring and nitrogen atmosphere, for 30 minutes. After cooling, the precipitated solid was filtered off and the filtrate was evaporated to dryness under reduced pressure. The oily residue solidified by treatment with an EtOH (0.2 ml) and H₂O (0.2 ml) mixture. The solid was collected, washed with EtOH/H₂O 1/1 and dried under vacuum at 40° C. to give 44 mg (50% yield) of product (yellow solid).

| | | | | | |
|---|---|---|---|---|---|
| Elemental | Calculated | C 54.68% | H 4.59% | N 6.07% | Br 17.32% |
| analysis | Found | C 55.93% | H 5.11% | N 5.41% | Br 16.36% |

¹H NMR (DMSO-d₆): δ 8.05 (1H, d, J=1.01 Hz); 7.79 (1H, d, J=8.29); 7.60 (1H, dd, J=1.01, 8.29); 4.39 (2H, q, J=6.98); 4.00 (3H, s); 3.71 (2H, s); 3.54 (4H, m); 2.41 (4H, m); 1.38 (3H, t, J=6.98).

### Example 5

### Ethyl 8-bromo-2-dimethylaminomethyl-3-methyl-4,5-dioxo-4,5-dihydro-3H-benzo[e]indole-1-carboxylate

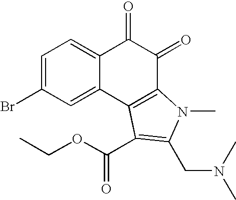

A solution of ethyl 8-bromo-2-bromomethyl-3-methyl-4,5-dioxo-4,5-dihydro-3H-benzo[e]indole-1-carboxylate of Example 2 (57 mg, 0.125 mmoles) in anhydrous THF (2 ml) was added, under stirring and nitrogen atmosphere, with a 2 M dimethyl amine solution in THF (125 μl, 0.25 mmoles). The resulting suspension was heated at 50° C., under stirring, for 30 minutes. The solid was filtered off and the filtrate was evaporated to dryness under reduced pressure. The semisolid residue was dissolved in absolute EtOH (0.4 ml) and the mixture was left under stirring at room temperature overnight. The precipitated solid was collected, washed with Et₂O and dried under vacuum at 40° C. to give 44 mg (yield 84% yield) of product (red solid).

| | | | | | |
|---|---|---|---|---|---|
| Elemental | Calculated | C 54.3% | H 4.57% | N 6.68% | Br 19.05% |
| analysis | Found | C 53.61% | H 4.61% | N 6.27% | Br 17.26% |

¹H NMR (DMSO-d₆): δ 8.05 (1H, d, J=1.78 Hz); 7.80 (1H, d, J=8.29); 7.60 (1H, dd, J=1.78, 8.29); 4.39 (2H, q, J=7.10); 3.98 (3H, s); 3.62 (2H, s); 2.19 (6H, s); 1.38 (3H, t, J=7.10).

### Example 6

### Ethyl 8-bromo-2-isopropylaminomethyl-3-methyl-4,5-dioxo-4,5-dihydro-3H-benzo[e]indole-1-carboxylate

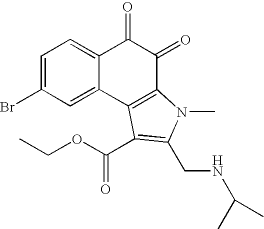

A solution of ethyl 8-bromo-2-bromomethyl-3-methyl-4,5-dioxo-4,5-dihydro-3H-benzo[e]indole-1-carboxylate of Example 2 (995 mg, 1.749 mmoles) and isopropyl amine (0.3 ml, 3.492 mmoles) in anhydrous toluene (40 ml) was heated at 50° C., under stirring and nitrogen atmosphere, for 4 hours. The solvent was evaporated to dryness under reduced pressure and the solid residue was washed with 2% NaHCO₃ (15 ml) and H₂O. The solid was then dried under vacuum at 40° C. and chromatographed on silica gel using CH₂Cl₂ and AcOEt as the eluent. The resulting solid was crystallized from AcOEt (2.8 ml) to give 617 mg (yield 56% yield) of product (red/orange solid).

| | | | | | |
|---|---|---|---|---|---|
| Elemental | Calculated | C 55.44% | H 4.89% | N 6.47% | Br 18.44% |
| analysis | Found | C 55.69% | H 4.91% | N 6.55% | Br 18.26% |

LC-MS: 433.0, MH⁺

¹H NMR (DMSO-d₆): δ 8.22 (1H, d, J=1.82 Hz); 7.79 (1H, d, J=8.08); 7.60 (1H, dd, J=1.82, 8.08); 4.38 (2H, q, J=7.12); 3.99 (3H, s); 3.86 (2H, s); 2.75 (1H, set, J=6.23); 1.86-1.76 (1H, s); 1.38 (3H, t, J=7.12); 1.02 (6H, d, J=6.23).

### Example 7

### Ethyl 8-bromo-3-tert-butoxycarbonylmethyl-2-methyl-4,5-dioxo-4,5-dihydro-3H-benzo[e]indole-1-carboxylate

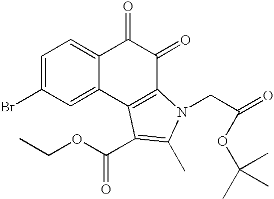

A suspension of ethyl 8-bromo-2-methyl-4,5-dioxo-4,5-dihydro-3H-benzo[e]indole-1-carboxylate of Example 1 (0.3 g, 0.8 mmoles), tert-butyl bromoacetate (0.3 ml, 1.8 mmoles) and K₂CO₃ (0.257 g, 1.8 mmoles) in dry DMF (10 ml) was heated at 60° C., under stirring and nitrogen atmosphere, for 3 hours. The solvent was evaporated off under reduced pressure and the residue was partitioned between H₂O (30 ml) and AcOEt (30 ml). The organic phase was separated, washed with H₂O (2×30 ml), dried over Na₂SO₄ and evaporated to dryness under reduced pressure. The residue was filtered on silica gel using hexane/AcOEt 1/1 as the eluent. The resulting oil was suspended in hexane (200 ml) and the mixture was left under stirring at room temperature for 4 days. The oil slowly solidified and the resulting solid was collected and washed with hexane to give 0.169 g (43% yield) of product (brown solid).

m.p. 105-108° C. (dec.)

| | | | | | |
|---|---|---|---|---|---|
| Elemental | Calculated | C 55.47% | H 4.66% | N 2.94% | Br 16.78% |
| analysis | Found | C 55.46% | H 4.67% | N 3.02% | Br 16.54% |

¹H NMR (DMSO-d₆): δ 8.35 (1H, d, J=1.74 Hz); 7.79 (1H, d, J=8.07); 7.63 (1H, dd, J=1.74, 8.07); 5.18 (2H, s); 4.39 (2H, q, J=7.11); 2.40 (3H, s); 1.45 (9H, s); 1.38 (3H, t, J=7.11).

### Example 8

### Ethyl 8-bromo-3-carboxymethyl-2-methyl-4,5-dioxo-4,5-dihydro-3H-benzo[e]indole-1-carboxylate

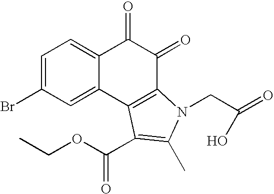

A solution of ethyl 8-bromo-3-tert-butoxycarbonylmethyl-2-methyl-4,5-dioxo-4,5-dihydro-3H-benzo[e]indole-1-carboxylate of Example 7 (0.14 g, 0.3 mmoles) and trifluoroacetic acid (2 ml) in anhydrous CH₂Cl₂ (10 ml) was kept at room temperature under stirring and nitrogen atmosphere for 5 hours and 30 minutes. The solvent was evaporated off under reduced pressure; the solid residue was suspended in Et₂O (5 ml) and the mixture was left under stirring at room temperature for 30 minutes. The solid was collected and washed with hexane to give 0.052 g (42% yield) of product (red solid).

m.p. 197-200° C. (dec.)

| | | | | | |
|---|---|---|---|---|---|
| Elemental | Calculated | C 51.45% | H 3.36% | N 3.33% | Br 19.01% |
| analysis | Found | C 50.38% | H 3.39% | N 3.27% | Br 18.04% |

¹H NMR (DMSO-d₆): δ 13.60-12.90 (1H, s); 8.34 (1H, d, J=1.69 Hz); 7.79 (1H, d, J=8.27); 7.63 (1H, dd, J=1.69, 8.27); 5.20 (2H, s); 4.39 (2H, q, J=7.11); 2.41 (3H, s); 1.38 (3H, t, J=7.11).

### Example 9

### Ethyl 8-bromo-3-carbamoylmethyl-2-methyl-4,5-dioxo-4,5-dihydro-3H-benzo[e]indole-1-carboxylate

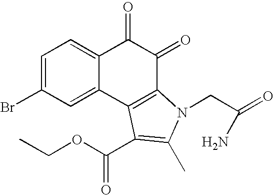

A suspension of ethyl 8-bromo-2-methyl-4,5-dioxo-4,5-dihydro-3H-benzo[e]indole-1-carboxylate of Example 1 (0.3 g, 0.8 mmoles), 2-bromoacetamide (0.124 g, 0.9 mmoles), K₂CO₃ (0.229 g, 1.6 mmoles) and KI (0.027 g, 0.16 mmoles) in dry DMF (10 ml) was kept at room temperature under stirring and nitrogen atmosphere for 7 hours 30 minutes. The mixture was diluted with H₂O (10 ml) and the solid was collected, washed with H₂O and dried under vacuum at room temperature. A suspension of the solid in absolute EtOH (50 ml) was refluxed, under stirring, for 1 hour. The suspension was hot filtered and the solid was collected and washed with hexane to give 0.081 g (23% yield) of product (brown solid).

m.p. >250° C.

| | | | | | |
|---|---|---|---|---|---|
| Elemental | Calculated | C 51.57% | H 3.61% | N 6.68% | Br 19.06% |
| analysis | Found | C 51.26% | H 3.53% | N 6.60% | Br 19.07% |

¹H NMR (DMSO-d₆): δ 8.33 (1H, d, J=1.73 Hz); 7.79 (1H, d, J=8.27); 7.71 (1H, s); 7.62 (1H, dd, J=1.73, 8.27); 7.33 (1H, s); 5.11 (2H, s); 4.39 (2H, q, J=7.09); 2.36 (3H, s); 1.37 (3H, t, J=7.09).

### Example 10

### Ethyl 5-bromo-2-methyl-1H-1,8,11-triazacyclopenta[1]phenanthrene-3-carboxylate

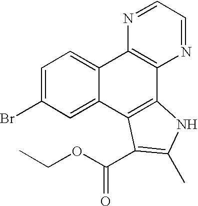

A suspension of ethyl 8-bromo-2-methyl-4,5-dioxo-4,5-dihydro-3H-benzo[e]indole-1-carboxylate of Example 1 (0.3 g, 0.8 mmoles), ethylenediamine (0.08 ml, 1.2 mmoles) and glacial AcOH (3 drops) in EtOH (20 ml) was refluxed, under stirring, for 5 hours. After cooling, the resulting solution was evaporated to dryness under reduced pressure and the solid residue was filtered on silica gel using 1/1 hexane/AcOEt as the eluent. The resulting solid was dried under vacuum at 40° C. to give 0.157 g (52% yield) of product (yellow solid).

m.p. 158-160° C. (dec.)

| | | | | | |
|---|---|---|---|---|---|
| Elemental | Calculated | C 56.27% | H 3.67% | N 10.94% | Br 20.80% |
| analysis | Found | C 55.78% | H 3.85% | N 10.28% | Br 18.52% |

LC-MS: 384.1, MH⁺

¹H NMR (DMSO-d₆): δ 13.25 (1H, s); 9.69 (1H, d, J=1.83 Hz); 9.09 (1H, d, J=8.76); 9.01 (1H, d, J=1.98); 8.98 (1H, d, J=1.98); 7.82 (1H, dd, J=1.83, 8.76); 4.41 (2H, q, J=7.07); 2.73 (3H, s); 1.43 (3H, t, J=7.07).

### Example 11

### Ethyl 5-bromo-2-methyl-1H-1,8,13-triazabenzo[a]cyclopenta[c]anthracene-3-carboxylate

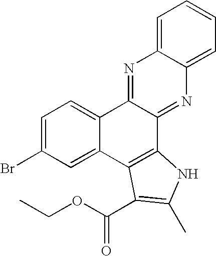

A suspension of ethyl 8-bromo-2-methyl-4,5-dioxo-4,5-dihydro-3H-benzo[e]indole-1-carboxylate of Example 1 (0.3 g, 0.8 mmoles), 1,2-phenylenediamine (0.134 g, 1.2 mmoles) and glacial AcOH (3 drops) in EtOH (20 ml) was refluxed, under stirring, for 7 hours. After cooling, the solid was collected and washed with Et₂O and hexane to give 0.313 g (87% yield) of product (yellow solid).

m.p. 234-235° C. (dec.)

| | | | | | |
|---|---|---|---|---|---|
| Elemental | Calculated | C 60.84% | H 3.71% | N 9.68% | Br 18.40% |
| analysis | Found | C 60.76% | H 3.70% | N 9.52% | Br 17.98% |

¹H NMR (DMSO-d₆): δ 13.29 (1H, s); 9.56 (1H, d, J=1.92 Hz); 9.21 (1H, d, J=8.67); 8.33 (1H, m); 8.28 (1H, m); 7.97 (2H, m); 7.84 (1H, dd, J=1.92, 8.67); 4.41 (2H, q, J=7.09); 2.73 (3H, s); 1.44 (3H, t, J=7.09).

### Example 12

Following procedures similar to those described in the above examples, the following compounds were prepared:
a) Ethyl 8-bromo-2-hydroxymethyl-3-methyl-4,5-dioxo-4,5-dihydro-3H-benzo[e]indole-1-carboxylate
b) Ethyl 8-bromo-2-diethylaminomethyl-3-methyl-4,5-dioxo-4,5-dihydro-3H-benzo[e]indole-1-carboxylate
c) Ethyl 8-bromo-2-methyl-3-benzyl-4,5-dioxo-4,5-dihydro-3H-benzo[e]indole-1-carboxylate
d) Ethyl 8-bromo-3-methyl-2-(4′-methylpiperazin-1′-yl)methyl-4,5-dioxo-4,5-dihydro-3H-benzo[e]indole-1-carboxylate
e) Ethyl 8-bromo-3-methyl-2-(piperazin-1′-yl)-methyl-4,5-dioxo-4,5-dihydro-3H-benzo[e]indole-1-carboxylate
f) Ethyl 8-bromo-3-methyl-2-(4′-(2-hydroxyethyl)-piperazin-1′-yl)-methyl-4,5-dioxo-4,5-dihydro-3H-benzo[e]indole-1-carboxylate
g) Ethyl 8-bromo-3-methyl-2-(4′-(2-aminoethyl)-piperazin-1′-yl)-methyl-4,5-dioxo-4,5-dihydro-3H-benzo[e]indole-1-carboxylate

### Example 13

### Primary Biochemical Assay for the Inhibition of Biot-Hif-1α⁷⁸⁶⁻⁸²⁶/GST-p300^{323/423}

The compounds were evaluated for their ability to inhibit the interaction between Hif-1α and p300 using a fluorescence assay (DELFIA™). The procedure described by Freedman S J at al., Nature Structural Biology 2003, 10 (7), 504-512 was suitably modified.

The compounds were obtained using the synthetic procedures described in the above examples.

The human biotinylated Hif-1α fragment corresponding to C-terminal 786-826 amino acids (Biotinylated Hif-1α⁷⁸⁶⁻⁸²⁶) was obtained from AnaSpec Inc (San Jose, Calif., USA) and used without further purifications.

A construct expressing the GST-p300³²³⁻⁴²³ fragment was transformed in the BL21 (DE3) strain of E. coli. Said construct was obtained by cloning in the expression vector pGEX-4T-1 (Amersham No. 27-45-80-01) the DNA sequence which encodes for the p300 region ranging from amino acids 323 to 423; the DNA sequence was obtained by PCR (Polymerase Chain Reaction). The protein region was induced with 1 mM isopropyl-1-tio-β-D-galactopyranoside (IPTG). The bacteria were lysed by sonication in the presence of a suitable buffer (50 mM Tris*HCl pH 8.00, 100 mM NaCl, 0.1 mM ZnSO₄, 1 mM DTT, 0.1 mg/ml lysozyme and a tablet of Complete EDTA-free Protease Inhibitor Cocktail Tablets (Roche, catalogue number 1 873 580)) and the GST fusion protein present in the soluble fraction was purified on a Glutathione-Sepharose 4B resin (Amersham Biosciences; no. 27-4574-01). The protein final concentration was determined according to Bradford with the Biorad assay (Bradford M., Anal. Biochem., 72, 248, (1976)). The purity of the sample was evaluated by SDS-PAGE. Samples were stored at −80° C. in 50% glycerol.

The assay was carried out using NUNC Maxisorp 96 wells plates as follows.

C96 NUNC Maxisorp plates (from Nunc, product No. 446612) were incubated overnight with streptavidin (Sigma; product No. S 4762) to a final concentration of 1 μg/ml in PBS buffer (Phosphate Buffered Saline 10 mM sodium phosphate, 150 mM sodium chloride pH 7.4). Each well was subsequently washed with 3×300 μl of TBST buffer (50 mM Tris*HCl pH 8.0, 150 mM NaCl, 0.05% (v/v) Tween 20). Each well was then added with 100 μl of a 10 nM solution of biotinylated Hif-1α⁷⁸⁶⁻⁸²⁶ in TBSB (50 mM Tris-HCl pH 8.0, 150 mM NaCl, 5% (w/v) BSA (Sigma, product No. A 2153)) and incubated for 1 h at 25° C. In the last row of each plate, only the TBSB buffer was added. Each well was subsequently washed three times with 300 μl of TBST buffer. The thus prepared plate represented the assay plate.

Separately, the plate (daughter plate) containing in each well 10 μl of each test compound dissolved in DMSO to a concentration of 10 μM was prepared. This plate was added with 100 μl of a 111 pM solution of GST-p300³²³⁻⁴²³ diluted in the incubation buffer (TBSB added with 0.1% (v/v) Tween 20, 0.5 mM DTT, 10 μM ZnCl₂) and the whole was mixed. 100 μl of the mixture contained in the daughter plate were immediately transferred to the assay plate.

Each daughter plate was prepared with 80 different compounds at a 10 μM concentration, safe for the two last well rows, wherein each well was added with 10 μl of DMSO. These two rows represented the positive (row 11, +Hif-1) and negative (row 12, −Hif-1) controls.

After incubation for 1 h at 25° C., each well was washed with 3×300 μl of TBST buffer (50 mM Tris-HCl pH 8.0. 150 mM NaCl, 0.05% (v/v) Tween 20). Each well was then added with 60.8 ng of an europium-labelled anti-GST antibody (DELFIA Eu-N1 labeled; Perkin Elmer; product no. A D 0251) dissolved in 100 μl of TBST buffer containing 10 μM ZnCl₂. After incubation for 1 h at room temperature, each well was washed with 3×300 μl of TBST buffer, then 100 μl of signal amplification solution (Enhancement Solution, Perkin Elmer product No. 1244-105) were added.

The plates were then read using a FUSION alpha-FP-HT reader (Perkin Elmer) in fluorescence mode for time resolution.

The activity of the compounds was calculated as follows. The fluorescence mean value of negative controls in row 12 of the plate test was subtracted from the fluorescence value of all the other wells. The resulting fluorescence value for each single well was then divided by the fluorescence mean value of positive controls in row 11 (which represented the maximum signal value, 100%) and expressed as percentage value. The inhibition value was expressed as the difference to 100 of the signal percentage calculated for each well.

Using daughter plates in which the compounds were present at 10 different concentrations ranging from 90 μM to 0.178 μM in each row, a dose-response curve could be calculated from which the IC₅₀ value was obtained (concentration of the compound necessary to inhibit the signal by 50%). Rows and 12 containing the vehicle only were the controls.

The IC₅₀ values obtained for some compounds of the present invention are reported in Table 1.

TABLE 1

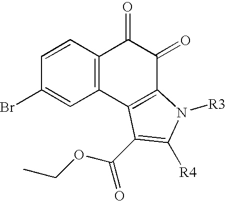

| | | | | IC₅₀ |
|---|---|---|---|---|
| Example | R3 | R4 | Chemical Name | (μM) |
| 6 | CH₃ | CH₂NH-iPr | Ethyl 8-bromo-2- | 9.1 |
| | | | isopropylaminomethyl-3-methyl-4,5- | |
| | | | dioxo-4,5-dihydro-3H- | |
| | | | benzo[e]indole-1-carboxylate | |
| 3 | CH₃ | CH₃ | Ethyl 8-bromo-2,3-dimethyl-4,5- | 3.3 |
| | | | dioxo-4,5-dihydro-3H- | |
| | | | benzo[e]indole-1-carboxylate | |
| 12(a) | CH₃ | CH₂OH | Ethyl 8-bromo-2-hydroxymethyl-3- | 14.2 |
| | | | methyl-4,5-dioxo-4,5-dihydro-3H- | |
| | | | benzo[e]indole-1-carboxylate | |
| 5 | CH₃ | CH₂N(CH₃)₂ | Ethyl 8-bromo-2- | 1.5 |
| | | | dimethylaminomethyl-3-methyl-4,5- | |
| | | | dioxo-4,5-dihydro-3H- | |
| | | | benzo [e]indole-1-carboxylate | |
| 12(b) | CH₃ | CH₂N(CH₂CH₃)₂ | Ethyl 8-bromo-2- | 13.3 |
| | | | diethylaminomethyl-3-methyl-4,5- | |
| | | | dioxo-4,5-dihydro-3H- | |
| | | | benzo[e]indole-1-carboxylate | |
| 1 | H | CH₃ | Ethyl 8-bromo-2-methyl-4,5-dioxo- | 13.2 |
| | | | 4,5-dihydro-3H-benzo[e]indole-1- | |
| | | | carboxylate | |
| 12(c) | CH₂Ph | CH₃ | Ethyl 8-bromo-2-methyl-3-benzyl- | 7.3 |
| | | | 4,5-dioxo-4,5-dihydro-3H- | |
| | | | benzo[e]indole-1-carboxylate | |
| 7 | CH₂COOCH(CH₃)₃ | CH₃ | Ethyl 8-bromo-3-tert- | 14.9 |
| | | | butoxycarbonylmethyl-2-methyl-4,5- | |
| | | | dioxo-4,5-dihydro-3H- | |
| | | | benzo[e]indole-1-carboxylate | |
| 4 | CH₃ | 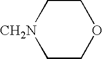 | Ethyl 8-bromo-3-methyl-2-(morpholin-4′-yl)methyl-4,5-dioxo-4,5-dihydro-3H-benzo[e]indole-1-carboxylate | 11.0 |
| 8 | CH₂COOH | CH₃ | Ethyl 8-bromo-3-carboxymethyl-2- | 32.9 |
| | | | methyl-4,5-dioxo-4,5-dihydro-3H- | |
| | | | benzo[e]indole-1-carboxylate | |
| 9 | CH₂CONH₂ | CH₃ | Ethyl 8-bromo-3-carbamoylmethyl- | 9.6 |
| | | | 3-methyl-4,5-dioxo-4,5-dihydro-3H- | |
| | | | benzo[e]indole-1-carboxylate | |

The compounds having inhibitory activity in the Hif-1α/p300 assay described above were evaluated using a cellular test on genetically modified human hepatocarcinoma Hep3B cells (Hep3B-VEGFLuciferase) in order to stably express a vector in which the Open Reading Frame of firefly Luciferase was under the control of the rat VEGF gene promoter.

Hif-1 Induction using deferoxamine (which induces hypoxia) induces luciferase transcription through activation of the VEGF promoter, which in turn causes an increase in luciferase activity, which can be measured with a commercially available kit. The compounds interfering with the Hif-1α/p300 complex inhibit Hif-dependent luciferase activation, resulting in a reduction of luciferase activity. This assay therefore allows to evaluate the activity of the compounds towards the VEGF promoter, which is essential to VEGF production and the subsequent tumor angiogenesis.

The Hep-3B-VEGFLuciferase cell line was obtained according to the following procedure.

Human hepatocarcinoma cells Hep-3B (ATCC reference No. HB-8064) were seeded onto 6 wells plates at a concentration of 2.5×10⁵ cells/well in 2 ml DMEM/10% FCS and the following day were transfected using Fugene 6 (Roche Biochemicals®). In each well, the transfection mixtures contained 6 μl of the transfection reagent Fugene 6, 1 μg of the pxp2-VEGF-luciferase reporter plasmid (VEGF rat promoter, NCBI GenBank No. of accession U22373, Levy et al., J. Biol. Chem. 270 (22), 13333-13340. 1995), and 10 ng of pcDNA3.1(+)plasmid (INVITROGEN) which makes cells resistant to neomycin. Transfection was carried out according to the manufacturer's instructions.

A suitable cell population (phenotypically resistant to neomycin) was selected by means of a cloning approach based on the “dilution limit” procedure (Sambrook J., Fritsch E. F. and Maniatis T. (1989) Molecular Cloning, A Laboratory Manual; Cold Spring Harbor Laboratories). The subsequent assays for Luciferase expression/activity “Luciferase assay” and for the quantification of secreted VEGF in the supernatant “ELISA secreted VEGF test”) were carried out with the stably transfected selected cells.

The following experimental protocol was used:

Day 1. Hep-3B-VEGF Luciferase cells were seeded onto 96-wells “blank” plates (a product by Greiner) at a density of 1×10⁴ cells/well/125 μl of medium, then left to adhere overnight in thermostat (37° C./5% CO₂).

Day 2. 75 μl of “3.2× working solutions” of compound (previously prepared in culture medium so that the DMSO concentration is 1.6% v/v) were added to the cells (partial volume/well=200 μl, partial concentration of compound=1.2×, partial concentration of DMSO=0.6%). After 1 hour incubation in thermostat, hypoxia was induced chemically by addition of 40 μl/well of a 6× (600 μM) stock solution of deferoxamine (final volume/well=240 μl, final concentration of compound=1×, final concentration of DMSO=0.5%, final concentration of deferoxamine=1×≈100 μM). The plates were then thermostated for further 18-20 hours.

Day 3. The “Luciferase assay” and the “secreted VEGF ELISA test” were carried out as follows.

“Secreted VEGF ELISA Test”

Quantification of secreted VEGF was performed using the kit “DuoSet Elisa Development System human VEGF” kit (R&D Systems). 100 μl/well of the supernatant from the “blank” 96 well-plates with the cells of the Hep3B/VEGF Luciferase clone were transferred into transparent 96-well plates of (Maxisorp) and assayed according to the indications of the kit manufacturer.

“Luciferase Assay”

Quantification of the expression of the Luciferase reporter gene was performed by means of the Bright Glo Reagent (Promega). After removing the supernatant and washing once with PBS, 40 μl/well of Bright Glo Reagent were added to blank 96-well plates with Hep3B/VEGF-Luciferase cells. The expression levels of the reporter gene were determined by reading the plates with a luminometer.

IC₅₀ values (concentration of the compound that causes 50% inhibition of the luciferase signal or 50% reduction of secreted VEGF) for some compounds of the invention are reported in table 2:

TABLE 2

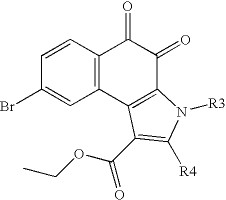

| | | | | IC₅₀ (μM) | |
|---|---|---|---|---|---|
| | | | | | Secreted |
| | | | | Luciferase | VEGF ELISA |
| Example | R3 | R4 | Chemical Name | assay | test |
| 6 | CH₃ | CH₂NH-iPr | Ethyl 8-bromo-2- | 0.30 | 0.30 |
| | | | isopropylaminomethyl- | | |
| | | | 3-methyl-4,5-dioxo- | | |
| | | | 4,5-dihydro-3H-benzo[e] | | |
| | | | indole-1-carboxylate | | |
| 3 | CH₃ | CH₃ | Ethyl 8-bromo-2,3- | 0.47 | 10.66 |
| | | | dimethyl-4,5-dioxo- | | |
| | | | 4,5-dihydro-3H-benzo[e] | | |
| | | | indole-1-carboxylate | | |
| 12(a) | CH₃ | CH₂OH | Ethyl 8-bromo-2- | 0.32 | 2.03 |
| | | | hydroxymethyl-3-methyl- | | |
| | | | 4,5-dioxo-4,5-dihydro- | | |
| | | | 3H-benzo[e]indole-1- | | |
| | | | carboxylate | | |
| 5 | CH₃ | CH₂N(CH₃)₂ | Ethyl 8-bromo-2- | 0.26 | 1.20 |
| | | | dimethylaminomethyl- | | |
| | | | 3-methyl-4,5-dioxo- | | |
| | | | 4,5-dihydro-3H-benzo[e] | | |
| | | | indole-1-carboxylate | | |
| 12(b) | CH₃ | CH₂N(CH₂CH₃)₂ | Ethyl 8-bromo-2- | 0.32 | 2.03 |
| | | | diethylaminomethyl-3- | | |
| | | | methyl-4,5-dioxo-4,5- | | |
| | | | dihydro-3H-benzo[e] | | |
| | | | indole-1-carboxylate | | |
| 1 | H | CH₃ | Ethyl 8-bromo-2- | 0.45 | 5.78 |
| | | | methyl-4,5-dioxo-4,5- | | |
| | | | dihydro-3H-benzo[e] | | |
| | | | indole-1-carboxylate | | |
| 12(c) | CH₂Ph | CH₃ | Ethyl 8-bromo-2- | 1.08 | 14.51 |
| | | | methyl-3-benzyl-4,5- | | |
| | | | dioxo-4,5-dihydro-3H- | | |
| | | | benzo[e]indole-1- | | |
| | | | carboxylate | | |
| 7 | CH₂COOCH(CH₃)₃ | CH₃ | Ethyl 8-bromo-3-tert- | 1.6 | 9.3 |
| | | | butoxycarbonylmethyl- | | |
| | | | 2-methyl-4,5-dioxo- | | |
| | | | 4,5-dihydro-3H-benzo[e] | | |
| | | | indole-1-carboxylate | | |
| 4 | CH₃ | 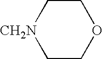 | Ethyl 8-bromo-3-methyl-2-(morpholin-4′-yl)methyl-4,5-dioxo-4,5-dihydro-3H-benzo[e]indole-1-carboxylate | 1.1 | 4.8 |
| 8 | CH₂COOH | CH₃ | Ethyl 8-bromo-3- | >12.5 | >12.5 |
| | | | carbossimethyl-2-methyl- | | |
| | | | 4,5-dioxo-4,5-dihydro- | | |
| | | | 3H-benzo[e]indole-1- | | |
| | | | carboxylate | | |
| 9 | CH₂CONH₂ | CH₃ | Ethyl 8-bromo-3- | 0.6 | >12.5 |
| | | | carbamoylmethyl-3- | | |
| | | | methyl-4,5-dioxo-4,5- | | |
| | | | dihydro-3H-benzo[e] | | |
| | | | indole-1-carboxylate | | |

### BIBLIOGRAPHY

1) Semenza G L, Nature Review Cancer, 3, Oct. 2003, 721-732.
2) Semenza G L. Trends Mol. Med. 2002 8:S62.
3) Semenza G L et al. J. Biol. Chem. 1996 271:17771.
4) Semenza G L et al. Am. J. Physiol. 1996 271:C1172.
5) Epstein A C et al. Cell 2001 107:43.
6) Semenza G L. et al. Genes Dev. 2001 15:2675.
7) Arany Z. et al. Proc. Nat. Acad. Sci. USA 1996 93; 12969.
8) Damert A. et al. Biochem J. 1997 327:419.
9) Eck M J. et al. Proc. Natl. Acad. Sci. USA, 2002 99:5367.
10) Wright P E et al. PNAS, 2002 99:5271.
11) Freedman, S. J. et al, Nature Structural Biology, 2003, 10(7), 504-12).
12) Zhong, H. et al., Cancer Research, 1999, 59, 5830-5.
13) Bos, R. et al., J. Nat. Cancer Inst. 2001, 93, 309-14.
14) Talks, K. I. et al., Am. J. Pathol., 2000. 157, 411-21.
15) Giatromanolaki, A. et al. Br. J. Cancer 85, 881-890 (2001).
16) Aebersold, D. M. et al. Cancer Res. 61, 2911-2916 (2001).
17) Birner, P. et al. Cancer Res. 60. 4693-4696 (2000).
18) Koukourakis, M. I. et al. Int. J. Radiat. Oncol. Biol. Phys. 53, 1192-1202 (2002).
19) Birner, P. et al. Clin. Cancer Res. 7, 1661-1668 (2001).
20) Birner, P. et al. Cancer 92, 165-171 (2001).
21) Koukourakis, M. I. et al. Cancer Res. 61, 1830-1832 (2001).
22) Zundel, W. et al. Genes Dev. 14, 391-396 (2000).
23) Hudson, C. C. et al. Mol. Cell. Biol. 22, 7004-7014 (2002).
24) Sodhi, A. et al. Cancer Res. 60. 4873-4880 (2000).
25) Mabjeesh, N. J. et al. Cancer Res. 62, 2478-2482 (2002).
26) Welsh, S. J. et al. Mol. Cancer Ther. 2, 235-243 (2003).
27) Mabjeesh, N. J. et al. Cancer Cell 3, 363-375 (2003).
28) Escuin, D. et al., Proceedings of the 95^{th} Annual Meeting of the American Association for Cancer Research, Abs. 5427.
29) Welsh, S. et al., Mol Cancer Ther. 2004; 3(3):233-244).
30) A. l. Kung et al., Cancer Cell, 6, 33-43 (2004)

1.-12. (canceled)

13. A method of treating an individual in need of having inhibited the interaction between transcription factor HIF-1α and its coactivator p300, comprising administering to the individual in an amount effective to treat the individual a compound of formula (I): 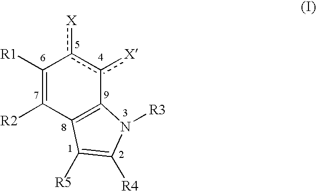
wherein [symbol]
is a single or double bond;
X and X′ are independently O, OH, NH, NH2, NH2OH;
or X and X′ are nitrogen and, together with the carbon atoms they are linked to, form a 6- or 10-membered heterocyclic or heteroaromatic ring;
R1 and R2, together with the atoms to which they are linked at 6- and 7-positions in formula (I), form a 6-membered aromatic or a 5- or 6-membered heteroaromatic ring;
R3 is hydrogen, acyl(C1-C4), (C1-C4)alkylsulfonyl, (C1-C4)alkylaminosulfonyl, straight or branched (C1-C4)alkyl, and may be interrupted by —O—, —S—, —N═, —NH—, —NHCONH—, —NHCOO—, —NHSO2NH—, —NHC(═NH)NH—, —NHC(═NH)—, —NHCSNH—, —CO—, —COO—, —CONH—, —SO2-, —SO2NH—, —CH═CH—, —C≡C— groups, and may be substituted with halogen, —NH2, —OH, —SH, —OCONH2, —COOH, —SO2NH2, —CONH2, —NHCONH2, —CN, phenyl, 5- or 6-membered heterocycle;
R4 is —NR6R7, wherein R6 and R7 are, independently, hydrogen, (C1-C4)acyl, (C1-C4)alkylsulfonyl, (C1-C4)alkylaminosulfonyl, straight or branched (C1-C4)alkyl, and may be substituted with halogen, amine, hydroxyl, thiol, carbamoyl, nitrile, phenyl or a 5- or 6-membered heterocyclic ring; —OR6; carbamoyl; straight or branched (C1-C4)alkyl, and interrupted by —O—, —S—, —N═, —NH—, —CO—, —COO—, —CONH—, —SO2-, —SO2NH— groups, and may be substituted with halogen, amine, hydroxyl, thiol, carbamoyl, nitrile, phenyl or a 5- or 6-membered heterocycle; up to 10-membered aromatic or heteroaromatic ring; 5- or 10-membered heterocyclic ring;
R5 is NH2; NR6R7; OR6; straight or branched (C1-C4)alkyl, and may be interrupted by —O—, —S—, —N═, —NH—, —CO—, —COO— groups, —CONH—, —SO2-, —SO2NH—, and may be substituted with halogen, amine, hydroxyl, thiol, carbamoyl, nitrile, phenyl or a 5- or 6-membered heterocyclic ring; up to 10-membered aromatic or heteroaromatic ring; a 5 or 6-membered heterocyclic ring; ureido;
the salts, isomers, enantiomers or diastereomers thereof.

14. The method of claim 13 in which R1 and R2 in the compound of formula (I) form a benzene ring or a benzene ring substituted with (C1-C4)acyl, (C1-C4)alkylsulfonylamino or (halogen) C1-C4alkyl, halogen, amine, mono or di(C1-C4)alkylamine, hydroxyl, (C1-C4)alkoxyl, thiol, (C1-C4)alkylthiol, carbamoyl, nitrile, sulfamoyl, phenyl.

15. The method of claim 13, wherein R1 and R2 in the compound of formula (I) form a benzene ring substituted with halogen.

16. The method of claim 13, wherein X and X′ in the compound of formula (I) are N and, together with the carbon atoms they are linked to, form a diazine or a benzodiazine.

17. The method of claim 13, wherein in the compound of formula (I) X═X′═O.

18. The method of claim 13, wherein in the compound of formula (I): X═X′═O;
R3 is selected from H, methyl, benzyl, carboxymethyl, tert-butoxycarbonylmethyl, carbamoylmethyl;
R4 is a methyl or ethyl group, and may be substituted with an hydroxy or amino group or with a primary or secondary amine;
R5 is ethoxycarbonyl.

19. The method of claim 13, in which the compound of formula (I) is selected from:
ethyl 8-bromo-2-methyl-4,5-dioxo-4,5-dihydro-3H-benzo[e]indole-1-carboxylate;
ethyl 8-bromo-2-bromomethyl-3-methyl-4,5-dioxo-4,5-dihydro-3H-benzo[e]indole-1-carboxylate;
ethyl 8-bromo-2,3-dimethyl-4,5-dioxo-4,5-dihydro-3H-benzo[e]indole-1-carboxylate;
ethyl 8-bromo-3-methyl-2-(morpholin-4′-yl)methyl-4,5-dioxo-4,5-dihydro-3H-benzo[e]indole-1-carboxylate;
ethyl 8-bromo-2-dimethylaminomethyl-3-methyl-4,5-dioxo-4,5-dihydro-3H-benzo[e]indole-1-carboxylate;
ethyl 8-bromo-2-isopropylaminomethyl-3-methyl-4,5-dioxo-4,5-dihydro-3H-benzo[e]indole-1-carboxylate;
ethyl 8-bromo-3-tert-butoxycarbonylmethyl-2-methyl-4,5-dioxo-4,5-dihydro-3H-benzo[e]indole-1-carboxylate;
ethyl 8-bromo-3-carboxymethyl-2-methyl-4,5-dioxo-4,5-dihydro-3H-benzo[e]indole-1-carboxylate;
ethyl 8-bromo-3-carbamoylmethyl-2-methyl-4,5-dioxo-4,5-dihydro-3H-benzo[e]indole-1-carboxylate;
ethyl 5-bromo-2-methyl-1H-1,8,11-triazacyclopenta[1]phenanthrene-3-carboxylate;
ethyl 5-bromo-2-methyl-1H-1,8,13-triazabenzo[a]cyclopenta[c]anthracene-3-carboxylate;
ethyl 8-bromo-2-hydroxymethyl-3-methyl-4,5-dioxo-4,5-dihydro-3H-benzo[e]indole-1-carboxylate;
ethyl 8-bromo-2-diethylaminomethyl-3-methyl-4,5-dioxo-4,5-dihydro-3H-benzo[e]indole-1-carboxylate;
ethyl 8-bromo-2-methyl-3-benzyl-4,5-dioxo-4,5-dihydro-3H-benzo[e]indole-1-carboxylate;
ethyl 8-bromo-3-methyl-2-(4′-methylpiperazin-1′-yl)methyl-4,5-dioxo-4,5-dihydro-3H-benzo[e]indole-1-carboxylate;
ethyl 8-bromo-3-methyl-2-(piperazin-1′-yl)-methyl-4,5-dioxo-4,5-dihydro-3H-benzo[e]indole-1-carboxylate;
ethyl 8-bromo-3-methyl-2-(4′-(2-hydroxyethyl)-piperazin-1′-yl)-methyl-4,5-dioxo-4,5-dihydro-3H-benzo[e]indole-1-carboxylate;
ethyl 8-bromo-3-methyl-2-(4′-(2-aminoethyl)-piperazin-1′-yl)-methyl-4,5-dioxo-4,5-dihydro-3H-benzo[e]indole-1-carboxylate.

20. The method of claim 13 for the treatment of a tumor or inhibiting metastasis.

21. The method of claim 20 for the treatment of lung carcinoma, breast carcinoma, prostate carcinoma, neuroblastoma, glioblastoma multiforme, melanoma, central nervous system tumors, oropharyngeal squamous cell cancer, cervical cancer, ovary, esophageal, kidney, colon, head-and-neck cancers or oligodendrioma.

22. A compound selected from:
ethyl 8-bromo-3-tert-butoxycarbonylmethyl-2-methyl-4,5-dioxo-4,5-dihydro-3H-benzo[e]indole-1-carboxylate;
ethyl 8-bromo-3-carboxymethyl-2-methyl-4,5-dioxo-4,5-dihydro-3H-benzo[e]indole-1-carboxylate;
ethyl 8-bromo-3-carbamoylmethyl-2-methyl-4,5-dioxo-4,5-dihydro-3H-benzo[e]indole-1-carboxylate;
ethyl 5-bromo-2-methyl-1H-1,8,11-triazacyclopenta[1]phenanthrene-3-carboxylate;
ethyl 5-bromo-2-methyl-1H-1,8,13-triazabenzo[a]cyclopenta[c]anthracene-3-carboxylate;
ethyl 8-bromo-3-methyl-2-(4′-methylpiperazin-1′-yl)methyl-4,5-dioxo-4,5-dihydro-3H-benzo[e]indole-1-carboxylate;
ethyl 8-bromo-3-methyl-2-(piperazin-1′-yl)-methyl-4,5-dioxo-4,5-dihydro-3H-benzo[e]indole-1-carboxylate;
ethyl 8-bromo-3-methyl-2-(4′-(2-hydroxyethyl)-piperazin-1′-yl)-methyl-4,5-dioxo-4,5-dihydro-3H-benzo[e]indole-1-carboxylate;
ethyl 8-bromo-3-methyl-2-(4′-(2-aminoethyl)-piperazin-1′-yl)-methyl-4,5-dioxo-4,5-dihydro-3H-benzo[e]indole-1-carboxylate.

23. A pharmaceutical composition comprising a compound according to claim 22 together with a pharmaceutically acceptable carrier or excipient.
